(19) United States
(12) Patent Application Publication
(10) Pub. No.: US 2018/0318098 A1
(43) Pub. Date: Nov. 8, 2018

(54) Coronal Correction Intervertebral Implant

(71) Applicant: WHIPPLE BETZ SPINE DEVELOPMENT PARTNERSHIP, Nashua, NH (US)

(72) Inventors: Dale Whipple, Nashua, NH (US); Randal Roberts Betz, Ocean City, NJ (US); Dustin Whipple, Mount Airy, MD (US)

(73) Assignee: WHIPPLE BETZ SPINE DEVELOPMENT PARTNERSHIP, Nashua, NH (US)

(21) Appl. No.: 15/404,112

(22) Filed: Jan. 11, 2017

(51) Int. Cl. A61F 2/44; A61B 17/70

(52) U.S. Cl. CPC A61F 2/442; A61B 17/7044

(57) ABSTRACT

This invention relates to an implant (20) and application thereof to correct coronal alignment in the vertebral column.

### CROSS REFERENCE TO RELATED APPLICATION

This application claims the benefit of application Ser. No. 62/387,762 filed Jan. 4, 2016.

### BACKGROUND OF THE INVENTION

### 1. Field of the Invention

A method for a coronal correction intervertebral implant

An apparatus for a coronal correction intervertebral implant

### 2. Description of the Prior Art

Surgical treatment for scoliosis, a three-dimensional deformity of the thoracic and/or lumbar spine, developed in the 20^{th} century. Instrumentation to assist with correction began to be developed in the 1950s with the Harrington rod and hook system [1]. While this was successful in moderately correcting the coronal deformity, it lacked the ability to correct rotation and the sagittal plane deformity . In the mid-1980s, new rod-hook multisegmented fixation systems began to be developed. This included the Luque segmental spinal instrumentation system [2], the Cotrel-Dubousset system [3], and the TSRH system [4].

Further significant advances developed in the 1990s and early 2000s with the use of pedicle screws attaching to rod systems. This allowed much more advanced correction of all three dimensions of the scoliosis deformity.

In the late 1970s, 80s, and 90s, anterior instrumentation for correction of spine deformity was promoted. These anterior techniques (including Zielke instrumentation [5]) involved removing the intervertebral discs, inserting bone graft into the discs, and/or using structural cages, vertebral body screws (either single or double), and rod systems.

All of these posterior and anterior systems were developed to correct the three dimensions of the spinal deformity and required fusion of all the instrumented vertebral segments.

### SUMMARY OF THE INVENTION

The invention provides for, displacing the vertebral bodies into vertical alignment as seen in the coronal plane.

The invention provides for, superior and inferior surfaces angled from parallel in the coronal view.

ADVANTAGES OF THE INVENTION

The invention in its broadest aspect utilizes an implant to correct spinal misalignment resulting in correction of the spine in the coronal plane.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is isometric view of the Implant

FIG. 2 is a view of the Spine and lateral curvature

FIG. 3 is a view of the Coronal Plane

FIGS. 4a and 4b show a front and side view of the Implant

FIG. 4a is Front View

FIG. 4b is Left Side View

FIG. 5 is the Implant between vertebral bodies

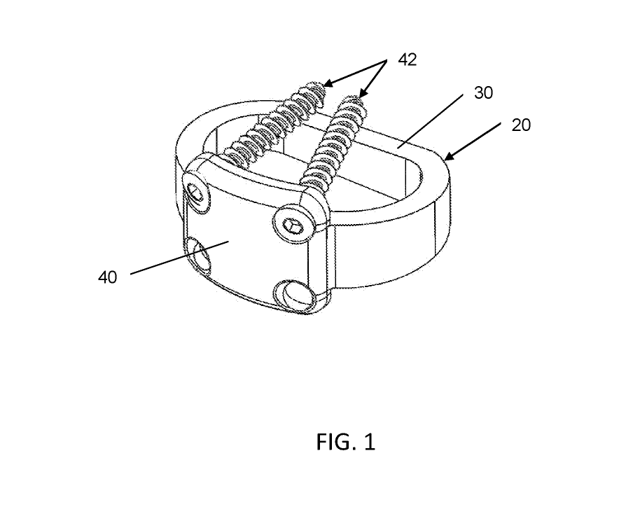

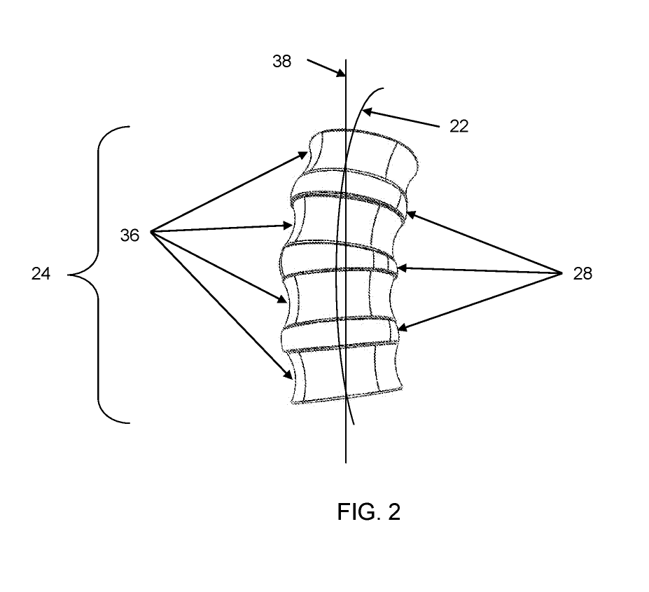

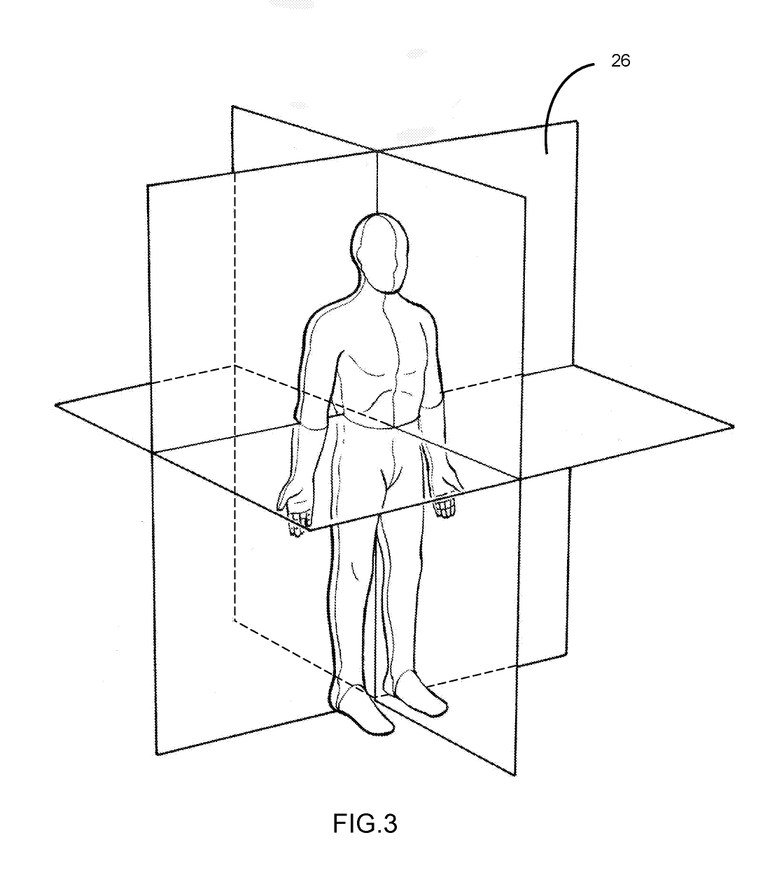

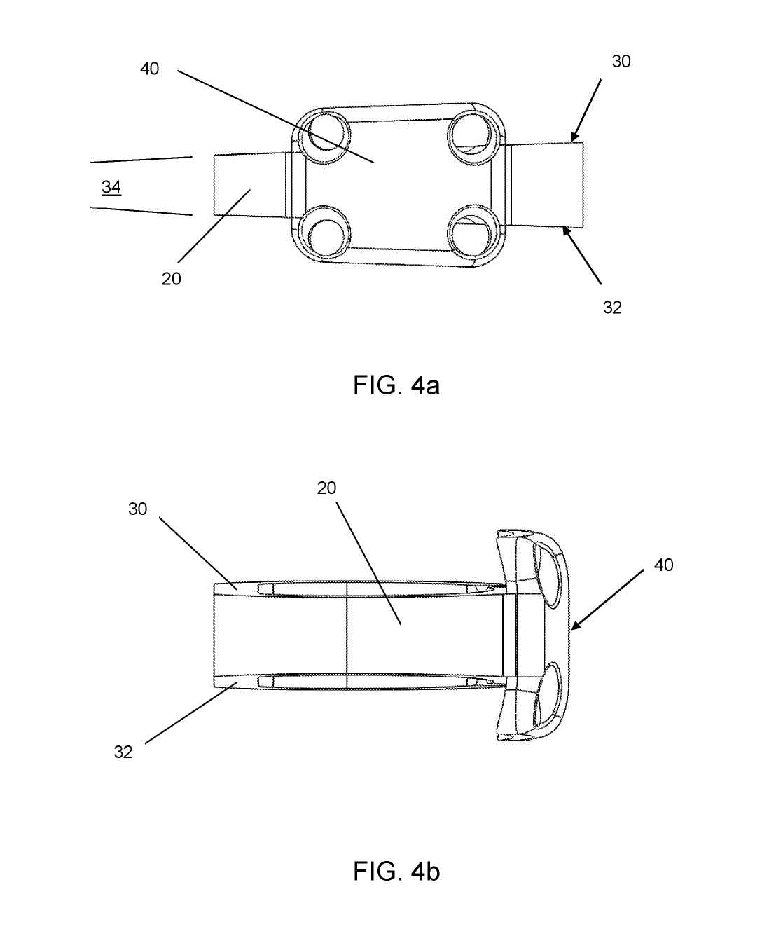

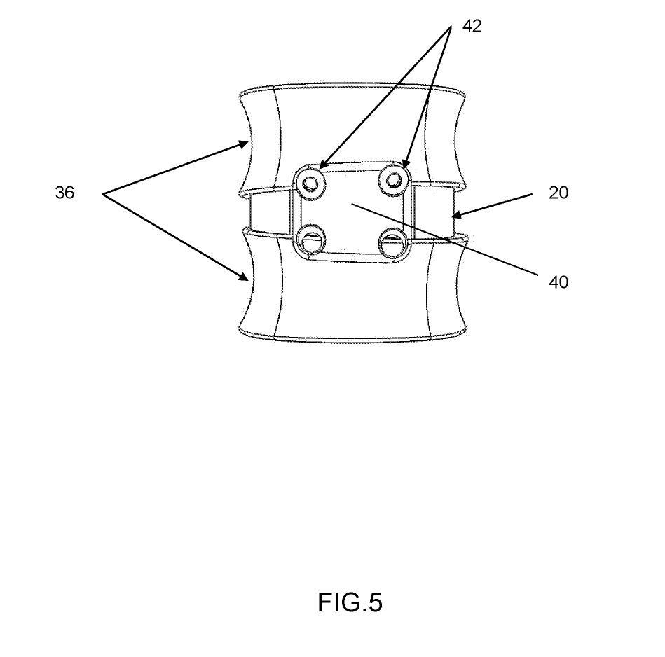

### DESCRIPTION OF THE ENABLING EMBODIMENT

Referring to the Figures, wherein like numerals indicate corresponding parts throughout the several views, a method for a coronal correction intervertebral implant (20),

To correct the lateral curve (22) of the spine (24), as seen in the coronal plane (26), the disc (28) is removed and replaced with an implant (20). The implant (20) superior surface (30) and inferior surface (32) are not parallel (34) in the coronal plane (26) resulting in the vertebral bodies (36) aligning on the vertical axis (38) of the spine (24). In the preferred embodiment the implant (20) has a plate (40) to allow screws (42) to stabilize the respective vertebral bodies (36).

Obviously, many modifications and variations of the present invention are possible in light of the above teachings and may be practiced otherwise than as specifically described while within the scope of the appended claims. That which is prior art in the claims precedes the novelty set forth in the “characterized by” clause. The novelty is meant to be particularly and distinctly recited in the “characterized by” clause whereas the antecedent recitations merely set forth the old and well-known combination in which the invention resides. These antecedent recitations should be interpreted to cover any combination in which the inventive novelty exercises its utility. The use of the word “said” in the apparatus claims refers to an antecedent that is a positive recitation meant to be included in the coverage of the claims whereas the word “the” precedes a word not meant to be included in the coverage of the claims. In addition, the reference numerals in the claims are merely for convenience and are not to be read in any way as limiting.

1. A method for a coronal correction intervertebral implant (20) including;
a solid device placed between adjacent vertebral bodies (36) to straighten the alignment as seen in the coronal plane (26) and characterized by, displacing the said vertebral bodies (36) substantially into vertical alignment (38) in said coronal plane (26).

2. An apparatus for a coronal correction intervertebral implant (20) comprising:
a solid device between said adjacent vertebral bodies (36) to straighten the alignment as seen in said coronal plane (26); and
said device having a superior surface (30) and an inferior surface (32) angled from parallel (34) as seen in the coronal plane (26).

3. Said apparatus for a coronal correction intervertebral implant (20) of claim 2 further comprising a hole that pierces both said superior surface (30) and said inferior surface (32).

4. A spinal surgical prosthesis implant (20) for placement between vertebral bodies (36) of a patient, the spinal surgical prosthesis implant (20) comprising:
a superior surface (30), an inferior surface (32);
said surgical prosthesis implant (20) configured to be surgically placed between two vertebral bodies (36) of a patient whereby said superior surface (30) and said inferior surface (32) are not parallel (34) one to the other in the coronal plane (26) of the patient whereby said spinal surgical prosthesis implant (20) corrects the lateral curve (22) of the vertebral bodies of the patient.

5. Said spinal surgical prosthesis implant (20) of claim 4 further comprising a plate (40) wherein said plate (40) is substantially parallel to the coronal plane (26) of the patient.

6. Said spinal surgical prosthesis implant (20) of claim 4 further comprising a plate (40) wherein said plate (40) is contoured to approximate the surfaces of the vertebral bodies (36) of the patient.

7. Said spinal surgical prosthesis implant (20) of claim 4 further comprising a plate (40) wherein said plate (40) and said device (20) are a single unit.

8. Said spinal surgical prosthesis implant (20) of claim 4 wherein said superior surface (30) and said inferior surface (32) are suitably contoured to approximate the vertebral bodies (36) contact surfaces.

9. Said spinal surgical prosthesis implant (20) of claim 4 further comprising:
a plate (40); and
a plurality of screws (42) configured to secure said superior surface (30), said inferior surface (32) and said plate (40) to the vertebral bodies (36).

10. Said spinal surgical prosthesis implant (20) of claim 4 wherein:
said superior surface (30) defining a superior surface plane;
said inferior surface (32) defining an inferior surface plane; and
said superior surface plane and said inferior surface plane are not parallel.

11. Said spinal surgical prosthesis implant (20) of claim 4 wherein said device (20) is a ring-shaped body defining a hole extending from said superior surface (30) to said inferior surface (32).

12. Said spinal surgical prosthesis implant (20) of claim 4 further comprising a plate (40) wherein said plate (40) is a generally planar shaped plate defining a plate surface plane extending generally perpendicular to the superior surface (30) and said inferior surface (32).

13. Said spinal surgical prosthesis implant (20) of claim 4 further comprising:
a plate (40); and
said plate (40) further comprises at least one through hole to receive at least one screw (42) whereby said at least one screw (42) stabilizes said spinal surgical prosthesis implant on the vertebral bodies (36) of the patient.

14. Said spinal surgical prosthesis implant (20) of claim 4 wherein:
said spinal surgical prosthesis implant (20) further comprises a plate (40);
said plate (40) is contoured to approximate the surfaces of the vertebral bodies (36) of the patient;
said plate (40) and said device (20) are a single unit;
said plate (40) is a generally planar shaped plate defining a plate surface plane extending generally perpendicular to the superior surface (30) and said inferior surface (32);
said spinal surgical prosthesis implant (20) further comprises a plurality of screws (42);
said plate (40) further comprises a plurality of through holes to receive said plurality of screws (42) whereby said plurality of screws (42) stabilize said plate (40) and said device on the vertebral bodies (36) of the patient;
said superior surface (30) and said inferior surface (32) are contoured to approximate the contact surfaces of the vertebral bodies (36);
said device is ring-shaped body defining a hole extending from said superior surface (30 to said inferior surface (32);
said superior surface (30) defining a superior surface plane;
said inferior surface (32) defining an inferior surface plane; and
said superior surface plane and said inferior surface plane are not parallel whereby said spinal surgical prosthesis implant (20) corrects the spinal misalignment of the spine in the coronal plane.
